(19) United States
(12) Patent Application Publication
(10) Pub. No.: US 2009/0061003 A1
(43) Pub. Date: Mar. 5, 2009

(54) CARRIERS FOR DRUG DELIVERY

(75) Inventors: Leif HERMANSSON, Molle (SE); Hakan ENGQVIST, Knivsta (SE)

Correspondence Address: YOUNG & THOMPSON, 209 Madison Street, Suite 500, ALEXANDRIA, VA, 22314, US

(73) Assignee: DOXA AB, Uppsala (SE)

(21) Appl. No.: 12/057,628

(22) Filed: Mar. 28, 2008

Related U.S. Application Data
(60) Provisional application No. 60/907,324, filed on Mar. 28, 2007.
(60) Provisional application No. 60/907,323, filed on Mar. 28, 2007.
(60) Provisional application No. 60/981,662, filed on Oct. 22, 2007.

(51) Int. Cl. A61K 9/14

(52) U.S. Cl. 424/489

(57) ABSTRACT

The present invention relates to chemically bonded ceramic precursor material of aluminates and silicates exhibiting a controlled release rate and properties that make the material suitable as a carrier material used in drug delivery. According to the invention, this is accomplished by selecting a microstructure based on pre-reacted phases and/or reacting phases, which contains the drugs. The present invention also relates to a cured ceramic material and a method of manufacturing said cured material. The precursor and the cured ceramic material according to the present invention can suitably be used for different types of drug intake and delivery.

### THE FIELD OF THE INVENTION

The present invention relates to ceramic-based carrier materials for drug delivery with controlled release time. The ceramics comprise chemically bonded ceramics The properties of the materials make them suitable as carrier material for different types of loading, delivery ways and medicaments. The carrier can also work as an implant material for site-specific drug administration, and as an injectable biomaterial. The invention also relates to a method of producing a carrier material loaded with a drug, as well as to a pharmaceutical composition comprising a drug.

### BACKGROUND

Carrier materials for drug delivery of pharmaceuticals are based on a broad range of materials, such as organic or inorganic polymers, metals and ceramics. The present invention comprises carrier materials based on ceramics.

General aspects of ceramics for use in drug delivery of drugs are given in by Ravaglioli et al. in J Mater Sci Mater Med. 2000 11(12):763-71 and by Lasserre and Bajpaj in Critical Reviews in Therapeutic Drug Carrier Systems, 15, 1 (1998).

Biocompatible cements based on calcium aluminate are e.g. described in the US patent application U.S. Pat. No. 7,244,301, “Heat generating biocompatible ceramic materials” (claiming priority from SE-0202895-9, filed Sep. 30, 2002). This document describes an implant material that could be used as carrier material in drug delivery. Another related patent application, WO2004058194, “Chemically bonded biomaterial with tailored properties”, generally discloses a biomaterial used in drug delivery.

In EP-1795171-A1, “Powdered CBC system with improved reaction feature”, the use of system as a carrier material is also presented.

In view of the prior art, there is a need for a carrier material for drug delivery that exhibits well-controlled microstructures, which lend the carrier material opportunities for selected and well-controlled release of the medicament. Issues regarding how and when the medicament is incorporated, where and when it is released, and how the medicament is released is the theme of this invention to assure high delivery safety for medicaments with regard to release pattern including time, rate, and amount of medicament delivered as well as safety aspects of the loaded carrier from chemical and mechanical point of view with regard to the patient, and further misuse/abuse aspects.

A controlled carrier material meeting the above mentioned criteria must also take account of and control the setting and curing reactions in vitro and in vivo, as well as to control the porosity of the finally cured material and use of additives and processing agents to assure an optimal microstructure.

### BRIEF DESCRIPTION OF THE INVENTION

The present invention provides materials based on ceramics, more specifically chemically bonded ceramics (CBC) and sintered ceramics and other additives that exhibit well-controlled microstructure enabling controlled and safe drug delivery, including aspects of dose dumping (due to occasionally too high release rate caused for example by inhomogenities or inclusions in the drug carrier material, or it could get stuck for example in the intestines) and potential abuse of highly potent medicaments.

According to one aspect, the present invention provides a drug carrier based on such a CBC material. The ceramic drug carrier material relates to both a ceramic precursor powder material and a cured ceramic material (the hydrated form) that at least in part have been made from said precursor material, and optionally by use of an inert additional ceramic material and other porous additives.

The ceramic material can be in the form of a stable precursor material and/or a chemically bound ceramic material, which is formed either before, just before or during the loading of the drug, or is finally developed after the material has been inserted/injected or during the release of the medicament. Depending on the composition (of non-hydrated, partly hydrated and/or fully hydrated material) of the carrier material, the chemically bound material may be formed one or more of the given instances.

In a further aspect, the present invention provides a method for producing a medicament-loaded carrier having optimised features in drug release. Such features are for example:

a) the chemical composition of the ceramic carrier material
b) the microstructure of the carrier material
c) the optional use of inert additives
d) the selection of medicament
e) safety against entrapment/dose dumping and abuse.

The present invention also includes the combined use of the carrier as an implant material. In one embodiment said carrier is used as an implant material. Before hardening, the carrier material according to the present invention exhibits a high degree of mouldability, including injectability. The injectability of the combined carrier/implant makes site-specific placement of the drug possible.

The carrier chemistry allows for loading of almost any medicament. The drugs can favorably be loaded in the water-liquid, in the pore system of inert filler particles and in processing agents (accelerators, retarders, viscosity controlling agents and other rheological agents). Thus drugs can be loaded both during formation of hydrates and/or after hydration by infiltration. The infiltration comprises water-penetration of precursor materials and/or hydrated materials using wetting at normal pressure, during vacuum, and/or overpressure. In one embodiment melting and infiltration of the drug is used. For hydrophobic medical agents, the agent can be easily mixed into the precursor powder and/or together with the second ceramic filler and other additives.

According to another aspect, the present invention provides pharmaceutical compositions comprising a medicament-loaded ceramic carrier, and optionally comprising a pharmaceutical acceptable buffer.

The composition can be a solid material (hydrated, partly hydrated or non-hydrated) or a suspension and is suitable for sublingual or oral intake, or subcutaneous or percutaneous injection.

### BRIEF DESCRIPTION OF THE DRAWINGS

FIG. 1 is a high-resolution TEM photo showing the microstructure of a cured ceramic Ca-aluminate material according to the present invention. In the photo, hydrate crystals having a size of 10-50 nm are seen. Specific surface area measurements (BET) of dried fully hydrated Ca-aluminate yield BET-values of >400 m²/g, corresponding to a hydrate size of approximately 25 nm, in accordance with the high-resolution TEM analysis.

FIG. 2 shows release curves for three materials based on chemically bonded ceramics indicating the possibility of obtaining slow almost constant release rate.

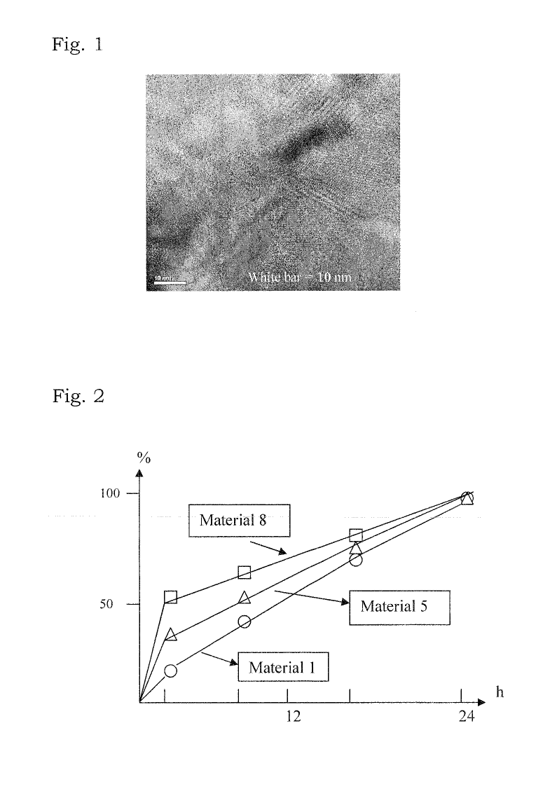

### DETAILED DESCRIPTION OF THE INVENTION

### The Drug Carrier

The present invention provides materials based on ceramics, chemically bonded ceramics (CBC) and additives such as sintered ceramics and other inert porous phases for release control by selection of precursor powder and/or inert powder, hydration solution including processing agents, and optionally pH control system and/or coating devices.

The precursor powder cures as a result of hydration reactions, between a ceramic oxide powder, primarily Ca-aluminates and/or Ca-silicates, and water. Through the hydration, new phases of hydrates are formed (crystalline and/or amorphous ones), which to a great part establish the microstructures needed to control the release of the drug incorporated in the precursor material. The hydration mechanism of these systems involves a reaction where the total volume of the precursor materials and the water (solution) is reduced. This allows a carrier to exhibit open porosity throughout its body even if a total low porosity in the interval 5-15% is selected. The microstructure developed consists of hydrates and nano-size channels, located between said hydrates, having a size of about 10-100 nm and 1-10 nm, respectively. Complementary porosity (porosity achieved by additives in the form of ceramic materials, etc) above 10 nm is according to the invention achieved by 1) partial hydration of the precursor material, 2) excess of water in the hydration step and 3) additional porous inert fillers, additional ceramics (such as hard particles and/or other hydrated or non-hydrated hydraulic phases) and other porous materials such as stable polymers and stable metals. The porosity size can thus be controlled in a carrier from 1-2 nm to micrometer size level, typically <10 micrometer.

The present invention presents a couple of unique reaction conditions related to the production of materials having a variety of microstructures. These include development of microstructures having different 1) type of porosity, 2) amount of porosity, 3) pore size and pore channel size, and 4) combination of different porosity structures.

Time and temperature for hydration is selected according to the invention with regard to drug and drug loading and to the selected release criteria. Temperature, as well as type of precursor powder, amount of precursor powder and processing agents, control the time selected for manufacturing the carrier. The manufacturing of the carrier can be according to the present invention done completely before/and/or during loading of drug, and/or during release of the drug. This renders according to the present invention a controlled release time to be selected from a few hours to days and months. Another aspect of the invention is a combined release with regard to time and amount.

The medicament can be of any kind. Preferable medicaments are those chosen from pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants (e.g., propantheline bromide, atropine sulfate, oxitropium bromide, timepidium bromide, scopolamine butylbromide, trospium chloride, butropiurn bromide, N-methylscopolamine methyl sulfate and methyloctatropine bromide); bronchodilators (e.g., theophylline, aminophylline, sodium cromoglicate); antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents (e.g., pravastatin sodium and fluvastatin sodium); sympathetic nervous stimulants (e.g., dihydroergotamine mesilate and isoproterenol hydrochloride, etilefrine hydrochloride); oral diabetes therapeutic drugs (e.g., glibenclamide, tolbutamide and glymidine sodium); oral carcinostatics (e.g., marimastat); contrast materials, radiopharmaceuticals, peptides, enzymes, vaccines and mineral trace elements or other specific anti-disease agents.

For highly potent medicaments such as opiodes and opioates (e.g. fentanyl and oxycodone), the present invention, also takes into account properties such as chemical stability, especially acid resistance, mechanical resistance (strength and crushing resistance) by selection of chemically bonded ceramics and other additives (sintered ceramics and stable polymers and metals) to prevent or heavily reduce the risk of abuse and to minimise the risk of dose dumping.

The medicaments that may be used with the present invention drug carrier/implant are not limited to the above given list of medicaments.

### Drug Loading, Manufacturing

The drug is introduced in the carrier by mixing the drug into the precursor powder, and/or the hydrated CBCs and/or other porous phases. The material is formed into a paste by mixing it with a water-based hydration liquid, which paste is then ready to be granulated by passing/extruding the paste through a patterned screen and then cut into granules. The granule size can according to the invention be selected within broad ranges. For oral intake a granule size above 30 micrometer is preferred to avoid possible dose dumping. The paste starts to develop the microstructure that to a great extent will contribute to the controlled release of the drug. The time and temperature after the mixing will determine the degree of hydration, i.e. the porosity obtained. The porosity can according to the invention be controlled within the interval 5-55% open porosity. The unique aspect by selecting CBCs is according to the invention that only open porosity is achieved even for the carriers with low porosity, 5-15%, or medium porosity 25-35%. See examples 1-3.

Depending on the type of drug delivery, including a combination of different drugs in the same carrier, for which the carrier material is intended, a combination of one or more of said techniques may be used.

Said water-based liquid may also comprise viscosity-controlling additives selected from one or more of carboxylic acids, polymerised carboxylic acids, thickening agents (starch and/or cellulose) or superplasticisers. These may be loaded with the drug before preparation of the final carrier.

The incorporation of the drug or medical agent into the carrier material in the second ceramic material, may be performed by filling the pores of the inert ceramic with said agents, by mixing it with the powder prior to mixing it with the hydration liquid, or mixing it with the hydration liquid prior to mixing it with the precursor powder. Depending on the type of drug delivery for which the carrier material is intended, a combination of one or more of said techniques may be used.

Optionally, it may also comprise a further inert phase of oxides, such as Ti, Si, Ba and/or Zr, in order to increase strength or radiopacity, if said property is desired. The oxides may take the form of porous and/or dense particles.

Other inert particles/additives that may work as additive ceramics according to the invention are based on very hard materials such as carbides and/or nitrides, which impart complementary properties to the final carrier system.

The carrier material may further comprise a third ceramic, including one or more of other hydrated or non-hydrated hydraulic phases, such as calcium phosphates, calcium sulphates, as well as hydroxyapatite.

The carrier material may further comprise a forth inert material comprised of a porous polymer and/or porous metal.

The inert additives may be porous or non-porous, but porous additives are normally preferred due to their ability to incorporate drugs in their open porosity, thus enabling the production of drug carriers having a more complex release profile.

To reduce the risk of abuse of highly potent drugs, the inert phases exhibit high hardness and wear resistance and high fracture toughness, and chemical resistance. It thus becomes difficult to extract any potent drugs having a high value on a possible criminal market.

### Controlling the Drug Release

The following properties are according to the present invention of significance with regard to the carrier for controlling drug release;
Type of ceramic precursor for producing the chemically bonded ceramic
Grain size distribution of the precursor powder particles
General microstructure of the material
Setting and curing time, and hydration temperature
The amount of water which is consumed in the curing reactions
pH-control
Microstructure of the additional particles for drug incorporation
Additives to ensure complementary porosity, an appropriate strength and/or
radio-opacity
Coating of carrier material
Drug loading aspects

### a) Type of Chemically Bonded Ceramic

The ceramics comprise chemically bonded ceramics (non-hydrated, partly hydrated or fully hydrated ceramics, or any combination thereof). The preferred chemical compositions, with an inherent property profile to meet the features described in the present invention, are those based on chemically bonded ceramics, which during hydration consume controlled amount of water. The preferred systems available are those based on aluminates and silicates, which both consume a great amount of water. Phases such CA2, CA, C3A and C12A7, and C2S and C3S in crystalline or amorphous state (C═CaO, A=Al₂O₃, SiO₂═S, according to common cement terminology) may be used. The pure aluminate and silicate phases are not available on the market. The Ca-aluminate and/or Ca-silicate phases may be used as separate phase or as mixtures of phases. The above-mentioned phases, all in non-hydrated form, act as the binder phase (the cement) in the carrier material when hydrated.

### b) Grain Size Distribution

The grain size of the precursor powder particles may be below 100 μm, preferably 1-20 μm. This is to enhance hydration. The precursor material is transformed into a nano-size microstructure during the hydration. This reaction involves dissolution of the precursor material and repeated subsequent precipitation of nano-size hydrates in the water (solution) and upon remaining non-hydrated precursor material. This reaction favourably continues until all precursor materials have been transformed and/or to a porosity determined by partial hydration using the time and temperature, as well as the H₂O in liquid and/or humidity, selected.

### c) General Microstructure of Material

Porosity generated during the hydration of the Ca-aluminates and Ca-silicates is open porosity due to the reaction mechanism, and may be in the interval of 5-55 vol.-%. The average pore channel size (i.e. the diameter of the pores formed between the particles of the hydrated material) may be 1-10 nm. The crystal size of the reacted hydrates is approximately 10-100 nm. When short hydration time and/or low amount of water of moisture at relative humidity>60%, is used, additional porosity is the result, with pore sizes in the interval 0.1-10 micrometer due to incomplete reaction.

### d) Setting and Curing Time, and Temperature

The setting time should be relatively short, below 30 minutes, and suitably in the interval of 5-15 minutes. The curing time and temperature are selected to produce controlled microstructure. The carrier materials are suitably hydrated at a temperature above 30° C., since this yields more stable hydrates (such as katoite and/or gibbsite) in the material, and thus a more stable material. The curing before loading and/or before introduction of the material into the body can be done in water and/or in an environment with high relative humidity (>60%). The setting and curing times and temperatures are of specific relevance when the carrier also works as an implant material.

e) The Amount of Water which is Consumed in the Curing Reactions

The water to cement ratio may be in the interval of 0.3-0.8. A ratio in the interval 0.4-0.5 is near complete hydration of the material without any excess of water. Excess water favours complementary porosity of the size lager than that formed by the hydrates, as does hydration in moisture at relative humidity>60%.

f) pH-Control

For medical agents sensitive to pH, the pH may be controlled in order to maintain their activity. A suitable pH is in the interval of 5-9. This is achieved by introduction of a buffer. The buffer may favourably according to the present invention be based on hydrogen-phosphates, and/or acid salts. Said buffer system may be included in the precursor powder or the hydration liquid, or both. This also reduces the risk of the carrier material to be dissolved during passage of the stomach. The pH-control also reduces the abuse risk, as a barrier against, for instance, acid dissolution of the material if having the intention of bringing the drug to the criminal market.

g) Microstructure of Additional Particles (Additives) for Drug Incorporation

The microstructure of the complementary additives which are penetrated by/loaded with the active medical agent is primarily characterised by its porosity, which should be an open porosity in the interval of 10-85 volume-%. The average pore size determined by Hg-porosimetry is in the interval of 0.1-10 μm. This is a complementary additive microstructure to that of main structure based on the chemically bonded ceramics. Examples of such additives according to the present invention include inert and hard ceramics such as oxides and/or carbides and/or nitrides. These phases yield a carrier material having increased strength and chemical resistance.

A third type of particles optionally incorporated are porous particles of other hydrated CBC's than those of Ca-aluminates and/or Ca-silicates, namely Ca-sulphates, and/or Ca-phosphates, as well as hydroxyapatite.

When producing a drug carrier having a porosity in the interval 0.1-10 μm, it is suitable to add stable polymers and acid resistant metals (generally inert and porous ceramics, polymers and metals).

The additional particles with a pore size in the interval 0.1-10 micrometer are introduced to speed up the release rate from slow release down to release time of a few hours (<5 hrs) and can favourably be used to be loaded with additional drugs for the rapid release.

h) Additives to Ensure an Appropriate Strength and/or Radiopacity

The CBC selected according to the present invention yields by itself a radio-opacity. In one embodiment of the invention, in order to impart a higher radio-opacity, additive phases with a high electron density are added to the carrier material. This allows the drug to be located in the body using X-ray techniques. These are favourably biocompatible phases. Examples of such phases are ZrO₂ and Sr- and Ba-containing glasses and/or salts thereof. The introduction of these phases also strengthens the materials and improves the safety due to their high strength, good mechanical properties. Some additives, such as ZrO₂, may also enable the carrier and drug to be traced due their radiopacity.

### i) Coat of the Precursor Material and the Drug

Further protection of the carrier and drug during passage through the stomach is obtained by coating of the precursor material. The coating may suitably be an acid-resistant and/or a hydrophobic layer. Coating of the precursor material and the hydrated material and/or the second ceramic phase(s) and/or the drug included, is preferably conducted if the medical agent is acid-sensitive.

### j) Drug Loading Aspects

The carrier chemistry allows for loading of almost any medical agent with a loading capacity of between <0.5 volume-% to as high as 20 volume-%.

The loading of the drug can be performed in several ways; the drug may be included, either partially or fully, in the powder (with non-hydrated and/or hydrated ceramics, the porous additives, sintered ceramics, stable polymers and/or metals) or in the hydration liquid comprising or not comprising any processing agents. The drug may be included one or more of these powders or liquids, and may be mixed with and/or incorporated into any open porosity of said components.

The ceramic material can be in the form of a stable precursor material and/or a ceramic material, which is formed before and/or just before and/or during the loading of the drug and/or finally developed after including and/or, during the release of the drug.

The drugs can favorably be loaded in the water-liquid, in the pore system of inert filler particles and in processing agents (accelerators, retarders, viscosity controlling agents and other Theological agents). Thus drugs can be loaded both during formation of hydrates and/or after hydration by infiltration. The infiltration comprises water-penetration of precursor materials and/or hydrated materials using wetting at normal pressure, during vacuum, and/or overpressure. In one embodiment melting and infiltration of the drug is used.

For hydrophobic medical agents, the agent can be easily mixed into the precursor powder and/or together with the second ceramic filler.

Additional drugs can separately be loaded in one or more of the different powder materials.

As mentioned above, the use of chemically bonded ceramics (CBCs) and porous additives for the loading of drugs can be executed in extremely many ways. Below it is summarised how a drug carrier according to the present invention, incorporating one or more drugs, may be produced.

The drug may basically be mixed with A) the powder (binder phase plus optional additives), B) the hydrating solution or a liquid additive, and C) the paste formed from combining A) and B)

Level A—Mixing with Powder
with unreacted CBCs
in porous additives (inert ceramics, stable polymer and metals, etc)
in solid processing agents (thickeners, viscosity-controlling agents, dispersing agents, cohesive agents)
in pre-reacted (hydrated CBCs), i.e. already incorporated into
a combination thereof.
   Level B—Mixing with Liquid
with aqueous hydration liquid
with liquid processing agents
a combination thereof.
   Level C—Mixing with Paste
with soft, not yet hardened, paste formed from mixing a powder according to A and a liquid according to B.

Different drugs can be placed in all parts of the carrier system (powder, solution, etc.) or in one or more places.

For porous additives and reacted dried hydrates (porous hydrates) different techniques to load the drug can be used, for example mixing, vacuum infiltration, and melting.

The carrier may be used as:

a pre-reacted hydrated material
a pre-loaded precursor material (CBCs and or porous additive)
paste
in granular form—hydrated or non-hydrated

In general, slow-release drugs are incorporated into the CBCs and the fast-release drugs are suitably incorporated into the additives or partly reacted CBCs.

### Pharmaceutical Compositions

The composition can be solid or a suspension for either sublingual and/or oral intake, and/or subcutaneous, and/or percutaneous injection. Non-active ingredients can be added. With non-active ingredients is meant water, alcohol, thickening agents, sweeteners, colours, antioxidants or other additives which may be useful for stabilizing the composition.

The ceramic carrier chemistry allows for loading of almost any medicament. The drugs can favorably be loaded in the water-liquid, in the pore system of inert filler particles and in processing agents (accelerators, retarders, viscosity controlling agents and other rheological agents). Thus drugs can be loaded both during formation of hydrates and/or after hydration by infiltration. The infiltration comprises water-penetration of precursor materials and/or hydrated materials using wetting at normal pressure, during vacuum, and/or overpressure. In one embodiment melting and infiltration of the drug is used. For hydrophobic medical agents, the agent can be easily mixed into the precursor powder and/or together with the second ceramic filler.

The carrier may be used as a vehicle for transport and delivery of the medicament as a paste in for example an implant. The combination of the material according to the present invention as carrier and implant material make site-specific placement of drugs and implants possible.

In summary, the release time is thus controlled mainly by the contents of the hydrated Ca-based cement phases, the higher the content of the cement, the longer the release time. The optimised (longest) release time is achieved for 100% of the hydrated phases with a water content close to the w/c required for complete hydration of the precursor Ca-aluminate and/or Ca-silicate. By introducing the optional additives, or by changing the w/c ratio, the release time can be controlled from a few hours to more than one day. The release time is also dependant upon where the drug is placed. In cortical bone, a release time of months seems possible.

The shorter release times are achieved when using a large excess of water (w/c), i.e. a ratio of more than 0.5, and the longer release times are obtained when using a ratio of 0.4-0.5, which is close to that of complete hydration without excess of water.

Using the mixed powder cement and an inert phase opens up for use of combined drugs, e.g. one for rapid release (based on the inert phase) and the other for slow release (based on the cement phase).

### EXAMPLES

The release time is strongly related to the microstructure of the carrier used. There are some important ways according to this invention, which are exemplified below.

### Example 1

The controlled porosity development requires well-defined phase composition. The sintering time and temperature for achieving C3A, C12A7 and CA are shown in Table 1. In this table is also included synthesis data for C3S and C2S.

TABLE 1
Synthesis data for some Ca-aluminate and Ca-silicate phases

| | Sintering temperature, | |
|---|---|---|
| Phase obtained | ° C. | Sintering time, (h) |
| C3A | 1325 | 3 |
| C12A7 (as glass phase) | 1350 | 4 + rapid cooling |
| CA | 1390 | 5 |
| C3S | 1350 | 4 |
| C2S | 1375 | 5 |

### Example 2

Applicable to both systems: the higher the Ca-content, the higher the reaction rate, (i.e. shorter time for porosity reduction is obtained). This is illustrated in Table 2 below. In Table 2 are described the porosity development of some specific selected phases of the Ca-aluminate and Ca-silicate systems as a result of selected hydration time and temperature. In all cases the mean particle size was close to 4 micrometer. The particle size was obtained by jet milling, and the particle size distribution determined by Malvern Mastersize 2000.

TABLE 2
Porosity development (Total porosity (TP) and nano-size porosity
(NP) in % for given hydration conditions (Temperature and Time).

| | W/c | Hydr | Hydr | | |
|---|---|---|---|---|---|
| Phase | ratio | Temp | Time | TP and (NP) | Comments |
| CA | 0.48 | 37 | 72 | (9.5) | No processing agents |
| | | 70 | 1 | (10) | No processing agents |
| | | 70 | 1 | (15) | % LiCl |
| | | 37 | 4 | 21 (11) | 0.2% LiCl |
| C3A | 0.40 | 37 | 3 | 20 (10) | No processing agents |
| | 0.42 | 60 | 2 | 15 (10) | 0.1% LiCl |
| C2S | 0.50 | 60 | 10 | 21 (15) | No processing agents |
| C3S | 0.51 | 60 | 5 | 20 (15) | No processing agents |

In a complementary study for CA where the w/c ratio was 0.48 and the temperature 70° C., the hydration took place in humid air, with RH close to 100%, a reaction time of 2 hrs, which resulted in a total porosity of 33% and an average pore size of approximately 1.5 micrometer. The hydration in a humid environment yielded hydrates of nano-size (nano-porosity) on the surface of the original particles of micrometer-size.

### Example 3

In Tables 3 and 4 below it is described how a complementary porosity, >0.1 micrometer, typically 1-10 micrometer, is obtained using a secondary ceramic pre-sintered ceramics. The selection of inert ceramics can be made from many oxides, nitrides and/or carbides. In the Table 3 is presented data using the following three ceramic materials, ZrO₂, SiC and Si₃N₄. These had approximately 20% open porosity, where the resulting porosity was determined by traditional water penetration technique.

TABLE 3
The inert ceramics

| Inert | | | Time, | Open porosity |
|---|---|---|---|---|
| ceramic | Type | Temperature, ° C. | (h) | in % |
| ZrO₂ | Mono-clinic | Sintering at 1380 | 5 | 21 |
| SiC | Alfa-silicon | Sintering at 1975 | 2 | 18 |
| | carbide | | | |
| Si₃N₄ | Reaction- | Nitridation at 1410 | 24 | 22 |
| | bonded | | | |

Hg-porosimetry revealed the interval for the pore distribution and the mean pore size given in Table 4.

TABLE 4
Pore size distribution of inert ceramics

| | % pores within | % pores within | |
|---|---|---|---|
| | the range 0.1-5 | the range 5-10 | |
| Inert ceramic | micrometer | micrometer | Mean pore size |
| ZrO₂ | 14 | 5 | 3.2 |
| SiC | 11 | 6 | 4.0 |
| Si₃N₄ | 9 | 11 | 6.8 |

### Example 4

In the following example it is presented how the materials in Examples 1-3 can be used as carriers for drug delivery yielding slow and close to constant release rate.

The compositions are summarised in Table 5. The test drug is a tartaric compound, N,N-6-trimethyl-2-p-tolylimidazo[1,2-a]pyridine-3-acetamide L-(+)-tartrate (2:1) but could be any other drug. The hydrating liquid was destined water. In all CA-cases the following process agents were used; 0.15% LiCl as an accelerator and 3% Methyl-cellulose. For the CS-cements, Ca-chloride was added.

TABLE 5
Composition in % (volume) of the specific materials
tested with regard to drug release.

| Material | CA | CS | ZrO₂ | SiC | Additives |
|---|---|---|---|---|---|
| 1 | 100 mono CA | — | — | — | Polycarboxylic |
| | | | | | compound, 4% |
| | | | | | molecular weight |
| | | | | | approx. 20000 |
| 2 | — | 100 C3S | — | — | CaCl₂, 15% |
| 3 | 70 mono-CA | — | 30 | — | 2% micro-silica, |
| | | | | | particle size 14 nm |
| | | | | | Polycarboxylic |
| | | | | | compound, 2.5% |
| 4 | 70 mono-CA | | | 30 | Polycarboxylic |
| | | | | | compound, 2.5% |
| 5 | — | 70 C3S | 30 | — | CaCl₂, 10% |
| 6 | 100 C3A | — | — | — | Polycarboxylic |
| | | | | | compound, 4% |
| 7 | 70 C3A | — | 30 | — | Polycarboxylic |
| | | | | | compound, 2.5% |
| 8 | 40 CA | — | 60 | — | Polycarboxylic |
| | | | | | compound, 1.5% |

The paste is formed via mixing of the precursor liquid and precursor powder at a powder to liquid ratio close to 4.3. The components added to the liquid imparted a high cohesiveness to the paste. This means that the paste is easily kept together during processing, thus avoiding e.g. phase separation. This also reduces the risk of uncontrolled spread of the paste into undesired voids when injected. The paste was easily granulated. In all of the cases above, the drug was mixed with the powder before hydration was initiated. Granulation was preceded by a step of homogeneously mixing the paste in a shaker, e.g. a Rotomix.

The amount of test drug was 4 mg per 150 mg total carrier when pure cement phases were used, and 8 mg per 150 mg for the cement compositions further comprising ZrO₂ or SiC (oxide and carbide, respectively).

Before the release tests, the material in granular form was hydrated in pure water at 60° C. for one hour. Thereafter, the materials were placed in a phosphate buffer solution at 37° C. for 2, 8, 16 and 24 hrs and the release of the drug was determined at each time point. The release was measured by HPLC.

Below is presented the time dependency of the slow release for Materials 1-8 as outlined in Table 5.

TABLE 6
The drug release with time (in mg)

| Release Time | | | | |
|---|---|---|---|---|
| Material | 2 h | 8 h | 16 h | 24 h |
| Material 1 | 0.5 | 1.3 | 2.8 | 3.9 |
| Material 2 | 0.6 | 1.4 | 2.8 | 4.0 |
| Material 3 | 2.7 | 3.9 | 5.8 | 8.0 |
| Material 4 | 2.7 | 3.7 | 5.9 | 8.0 |
| Material 5 | 2.8 | 4.0 | 5.9 | 8.0 |
| Material 6 | 0.5 | 1.3 | 2.7 | 3.8 |
| Material 7 | 2.6 | 3.8 | 5.9 | 8.0 |
| Material 8 | 4.5 | 5.5 | 6.9 | 8.0 |

From the Table 6 it is clearly demonstrated the relatively rapid release from the oxide and the carbide (Examples 3-5 and 7-8), while the pure chemically bonded phases (Examples 1-2 and 6) contributes to a prolonged release time. The pure phases almost have a constant release, which is even more evident when presenting the release results as time curves in FIG. 2 for Materials 1, 5 and 8). The pure chemically bonded phases exhibit an almost constant release, while the composite materials exhibit a two-step behaviour; first rapid release and then a slow almost constant release rate. This also provides possibilities for loading drugs with different release requirements—one with fast release within a few hours loaded preferably in the porous inert ceramic, metal or polymer material, and a second drug in the chemically bonded ceramic material for long term effect.

1. A non-hydrated ceramic drug carrier or implant material for drug delivery comprising:
10-100% of a powdered hydraulic non-hydrated ceramic binder phase selected from one or both of calcium aluminate and calcium silicate, comprising one or more of the phases selected from the group consisting of CA2, CA, C12A7, C3A, C2S and C3S, and
optionally additives,
wherein the drug carrier/implant exhibits an open porosity of 5-55 volume %.

2. The non-hydrated ceramic drug carrier or implant material according to claim 1, wherein the additive is selected from fully hydrated ceramic sulphate or ceramic phosphate, or hydroxy apatite, a combination thereof.

3. The non-hydrated ceramic drug carrier or implant material according to claim 1, wherein the additive is selected from fully hydrated calcium sulphate or calcium phosphate, or hydroxy apatite, a combination thereof.

4. The non-hydrated ceramic drug carrier or implant material according to claim 1, wherein the additive is a biocompatible inert phase selected from one or more of oxides, carbides, nitrides.

5. The non-hydrated ceramic drug carrier or implant material according to claim 4, wherein the additive is selected from SiC and Si₃N₄.

6. The non-hydrated ceramic drug carrier or implant material according to claim 4, wherein said inert phase is selected from one or more of the porous or non-porous oxides of Ti, Si, Ba and Zr.

7. The non-hydrated ceramic drug carrier or implant material according to claim 4, wherein said inert phase is selected from one or more of porous polymers and porous metals.

8. The non-hydrated ceramic drug carrier or implant material according to claim 4, wherein said inert phase is selected from glass containing one or more of ZrO₂, Sr and Ba or their salts.

9. The non-hydrated ceramic drug carrier or implant material to claim 1, wherein the additive is selected from one or more of non-aqueous retarders, accelerators, dispersants or viscosity controlling agents and buffers.

10. The non-hydrated ceramic drug carrier or implant material according to claim 9, wherein the viscosity-controlling agents are selected from carboxylic acids, poly-carboxylic acids, starch, cellulose and superplasticisers.

11. The non-hydrated ceramic drug carrier or implant material according to claim 1, wherein the binder phase comprises 80-100% calcium aluminate.

12. The non-hydrated ceramic drug carrier or implant material according to claim 1, wherein the material is constituted by compacted granules having a granule size of 30-500 μm.

13. The non-hydrated ceramic drug carrier or implant material according to claim 1, further comprising one or more drugs mixed with the binder phase or incorporated into any additive, or a combination thereof.

14. The non-hydrated ceramic drug carrier or implant material according to claim 13, wherein the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

15. A fully hydrated ceramic drug carrier or implant material for drug delivery based on the non-hydrated carrier or implant material defined in claim 1, in hydrated form, wherein the drug carrier/implant exhibits a crystal size of the hydrates of approximately 10-100 nm, an open porosity of 5-55 volume %, and a pore channel size of 1-10 nm between the hydrate crystals.

16. The fully hydrated ceramic drug carrier or implant material according to claim 15, comprising one or more of katoite and gibbsite.

17. The fully hydrated ceramic drug carrier or implant material according to claim 15, wherein the carrier or implant is coated with an acid-resistant layer.

18. The fully hydrated ceramic drug carrier or implant material according to claim 15, further comprising one or more drugs incorporated into the hydrated binder phase or incorporated into any additive, or a combination thereof.

19. The non-hydrated ceramic drug carrier or implant material according to claim 18, wherein the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

20. The fully hydrated ceramic drug carrier or implant material according claim 15, further comprising a phosphate or phosphate/saline buffer.

21. A partly hydrated ceramic drug carrier or implant material for drug delivery based on a mix of the non-hydrated carrier or implant material defined in claim 1 and a hydrated carrier or implant material in hydrated form, wherein the drug carrier/implant exhibits a crystal size of the hydrates of approximately 10-100 nm, an open porosity of 5-55 volume %, and a pore channel size of 1-10 nm between the hydrate crystals, and the hydrated parts exhibit a crystal size of the hydrates of approximately 10-100 nm and a pore channel size of 1-10 nm between the hydrate crystals, and the overall material has an open porosity of 5-55 volume %.

22. The partly hydrated drug carrier or implant material according to claim 21, comprising one or more of katoite and gibbsite.

23. The partly hydrated drug carrier or implant material according to claim 21, wherein the carrier is coated with an acid-resistant layer.

24. The partly hydrated ceramic drug carrier or implant material according to claim 21, further comprising one or more drugs mixed with the powdered binder phase or additives, incorporated the hydrated binder phase, or incorporated into any additive, or a combination thereof.

25. The partly hydrated ceramic drug carrier or implant material according to claim 24, wherein the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

26. The partly hydrated ceramic drug carrier or implant material according to claim 21, wherein the non-hydrated parts are in the form of a loose powder or compacted granules having a granule size of 30-500 μm, or a combination thereof.

27. A method for producing the non-hydrated ceramic drug carrier or implant material defined in claim 1, comprising the steps of:
mixing 10-100% of a powdered hydraulic non-hydrated ceramic binder phase selected from one or both of calcium aluminate and calcium silicate, comprising one or more of the phases selected from the group consisting of CA2, CA, C12A7, C3A, C2S and C3S and optionally additives and optionally drugs;

28. The method according to claim 27, wherein the compaction step is preceded by a step of freeze-drying or spray-drying the binder phase or any additives.

29. The method according to claim 27, wherein one or more drugs are mixed with the binder phase or are added as drugs incorporated into an additive, or a combination thereof.

30. The method according to claim 29, wherein the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

31. The method according to claim 27, further comprising the steps of compacting the binder phase and any additives into a raw compact and granulating said raw compact into granules having a size of 30-500 μm.

32. A method for producing the fully hydrated ceramic drug carrier or implant material defined in claim 15, comprising the step of:
mixing the non-hydrated material defined in claim 1, an aqueous hydration liquid, and optionally additives, and optionally drugs, such that a paste is formed; and
allowing the paste to harden.

33. The method according to claim 32, wherein the paste, while still soft, is granulated to a granule size of 30-500 μm.

34. The method according to claim 32, wherein the hydration temperature is kept above 30° C.

35. The method according to claim 32, wherein a drug is mixed with the carrier material using wetting at one or more of pressure conditions selected from ambient pressure, vacuum, and overpressure.

36. The method according to claim 32, wherein one or more drugs are incorporated via mixing with the non-hydrated material, or via addition of additives incorporating said drugs, or via mixing with the hydration liquid or liquid additive, or a combination thereof.

37. The method according to claim 36, wherein the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

38. The method according to claim 32, further comprising adding a phosphate or phosphate/saline buffer.

39. A method for producing the partly hydrated ceramic drug carrier or implant material defined in claim 21, comprising the step of:
mixing the non-hydrated material defined in claim 1 and the a fully hydrated material defined in claim 15 in hydrated form, wherein the drug carrier/implant exhibits a crystal size of the hydrates of approximately 10-100 nm, an open porosity of 5-55 volume %, and a pore channel size of 1-10 nm between the hydrate crystals, and optionally additives and optionally drugs.

40. A method according to claim 39, further comprising one or more drugs mixed with the non-hydrated material or additives, incorporated into the hydrated binder phase, or incorporated into any additive, or a combination thereof.

41. A method according to claim 40, wherein the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

42. A pharmaceutical composition for controlled release comprising one or more of the non-hydrated, fully hydrated or partly hydrated drug carrier or implant materials defined in claim 1, and optionally non-active ingredients and optionally one or more drugs.

43. The pharmaceutical composition according to claim 42, where the drug is selected from therapeutic agents including pain relief drugs including highly potent medicaments, antiphlogistics, drugs for cancer/tumour treatment, vascular treatment, bone restoration, antibacterial and anti-inflammatory agents, antifungal agents, antivirus agents, analgesics, anticonvulsants; bronchodilators; antidepressants, auto-immune disorder and immunological disease agents, hormonal agents, TGB-beta, morphogenic protein, trypsin-inhibitor, osteocalcine, calcium-binding proteins (BMP), growth factors, Bisphosphonates, vitamins, hyperlipidemia agents; sympathetic nervous stimulants; oral diabetes therapeutic drugs; oral carcinostatics; contrast materials, radiopharmaceuticals, peptides, enzymes, vaccines and mineral trace elements or other specific anti-disease agents.

44. (canceled)
